(19) United States
(12) Patent Application Publication
(10) Pub. No.: US 2025/0235622 A1
(43) Pub. Date: Jul. 24, 2025

(54) SYRINGE ASSEMBLY WITH MARKING ON PLUNGER FOR DOSING

(71) Applicant: SJJ Solutions B.V., 's-Gravenhage (NL)

(72) Inventors: Joost SCHOOTSTRA, Delft (NL); Sander SCHOOTSTRA, Rijswijk (NL); Jasper SCHOOTSTRA, Oegstgeest (NL)

(21) Appl. No.: 18/856,260

(22) PCT Filed: Apr. 13, 2023
(86) PCT No.: PCT/NL2023/050194

§ 371 Date: Oct. 11, 2024

(30) Foreign Application Priority Data
Apr. 13, 2022 (NL) 2031567

(51) Int. Cl. A61M 5/315; A61M 5/31; A61M 5/34

(52) U.S. Cl. CPC A61M 5/315; A61M 5/3135; A61M 5/34; A61M 2205/583

(57) ABSTRACT

A syringe assembly is provided, comprising a barrel comprising a barrel body with an axial passage therethrough, wherein the barrel body is at least partially transparent and the barrel comprises at least one marking. The assembly further comprises a plunger with a visible marking connected to or provided on a plunger body, wherein the visible marking is positioned at a distance of a piston of the plunger. The marking of the plunger is visible through at least part of the barrel body. Aligning a marking on the plunger body with the marking of the barrel allows for accurate dosing of liquid.

### TECHNICAL FIELD

The present disclosure relates to the field of syringe assemblies, comprising a barrel and plunger with a piston at a distal end of the plunger.

### BACKGROUND

Liquids, such as liquid medicine, are often provided in a syringe assembly for providing a pre-determined amount of the liquid to for example a body part of a patient using a needle. For many liquid medicines, a specific volume has to be administered. For example in the field of ophthalmology, the volume of liquid medicine used has to be accurately measured, for example in the order of 0.05 mL or even less. A syringe assembly typically comprises a barrel and a plunger, which plunger can be moved through the barrel. At a distal end of the plunger, a piston is provided, which creates a seal with an inner wall of the barrel. A piston may also be referred to as a stopper.

To allow a user to determine the volume of liquid medicine inside the syringe assembly, one or more markings may be provided on the barrel. Since the volume of liquid medicine inside the syringe assembly is determined by a position of the piston inside the barrel of the syringe assembly, a distal end of the piston can be aligned with a marking on the barrel in order to determine the volume of liquid medicine inside the syringe assembly. To allow for convenient visual identification of the piston inside the barrel, the piston is typically provided in a darker colour than the plunger body to which the piston is connected. For example, the piston can be coloured black, whereas the plunger body can be white or even transparent.

### SUMMARY

It has been observed that, in particular when the volume of liquid inside a syringe assembly is small, for example in the order of millilitres or even tenths of millilitres, a small displacement of a piston inside a barrel of the syringe assembly may result in a significant relative change of the volume of liquid inside a syringe assembly. This may for example be caused by a practical minimum of an inner diameter of a barrel, for example due to manufacturing constraints, and/or prescribed minimal volumes of a syringe, e.g. 1 mL, for example according to industry standards, and/or a minimum size a syringe has to have in order to be properly handled.

Furthermore, it has been observed that it may be difficult for a user to align a piston with a marking on the barrel, in particular when the distal end of the piston does not form a flat plane and/or when a part of the distal end of the piston which creates a seal with an inner wall of the barrel is not the outermost distal end of the piston—for example when the piston has a curved or convex end-plane.

Even further, it has been observed that a skirt part of the barrel at a distal end of the barrel may prevent or make it more difficult to print a marking, in particular a zero marking, at or near the distal end of the barrel. However, use of such a skirt part may be preferred, for example in order to connect a needle hub to the distal end of the barrel with a threaded connection, such as a known Luer-lock.

In case a needle hub is fitted to the barrel, for example using a Luer-slip connection, a marking at or near the distal end of the barrel may be covered by the needle hub. This may make it difficult or even impossible for a user to see this marking, and/or to align a piston with such a marking.

In general, it has been further observed that a distal end of a barrel may be tapered towards the distal end of the barrel—for example to comply with well-known Luer-lock or Luer-taper standards. Printing a marking, such as a zero-marking, on such a tapered part of the barrel may be technically complex. This may make printing on the tapered part economically unfeasible.

It is an object to increase the precision and/or accuracy with which a user can determine a volume of liquid present inside a syringe assembly, in particular when this volume is determined visually. As such, it is an object to increase precision and/or accuracy with which a user can dose a volume liquid.

A first aspect provides a plunger for a syringe assembly, the plunger comprising a plunger body with a piston at or near a distal end of the plunger, a visible marking connected to or provided on the plunger body, wherein the visible marking is positioned at a distance of the piston. The plunger is generally arranged to have the plunger body inserted into a barrel with the piston first.

Typically, part of the plunger body extends out of a proximal end of the barrel, allowing the user to press the plunger further into the barrel and/or to pull the plunger away from the distal end of the barrel.

Generally, a plunger is formed by a plunger body and a piston. Furthermore, in general, the plunger body and the piston may be separate elements, which may be connected to each other, for example via a threaded or fitted connection, or may even be separate elements in use.

When the plunger body and the piston are separate elements, the plunger body and the piston may in use at least move together, for example when the plunger is inserted into the barrel towards the distal end of the barrel. In such cases, the plunger body may be used to push the piston further into the barrel, for example for expelling liquid from the barrel, regardless of whether the plunger body and the piston are connected or not.

Alternatively, the plunger body and the piston may be formed as a single body, for example in an injection moulding process using a single material or multiple types of material. The plunger body itself may be a single body, or may comprise multiple connected bodies.

Because the visible marking is positioned at a distance of the piston, the piston of the plunger no longer has to be used as a visible marking which has to be aligned with a marking of a barrel. In other words, the marking may be positioned at a distance from the part of the plunger, for example the piston, which forms an essentially liquid-tight seal with the barrel. As a result hereof, for example, the piston of the plunger can have any shape, including a curved, convex, or even concave shape, without making it more difficult for a user to measure a specific volume of liquid inside the barrel. It has been observed that when a curved, convex, or even concave shape has to be aligned with a marking on a barrel, it may be difficult for a user to determine which part of said curved, convex, or even concave shape has to be aligned with the marking—e.g. a minimum or a maximum of said shape.

Since the piston does not have to be used anymore as a marking to be aligned with a marking on the barrel, more freedom may be provided as to how the marking of the plunger can be shaped and sized. This in turn may allow for more accurate dosing, because the freedom on how the marking of the plunge can be shaped and sized may now be independent of a shape and/or size of the piston.

The visible marking of the plunger is in use visible through the transparent part of a barrel body, and is as such visible to a user. When the marking of the plunger is visible through the barrel body, the user may align the marking of the plunger with a marking of the barrel to ensure a desired volume of liquid is present inside the barrel, or that essentially no volume of liquid is present inside the barrel. Preferably, the marking of the plunger is a relatively thin line, with a thickness for example smaller than 10% of an axial length of the plunger, or even smaller than 1% of said axial length.

When the visible marking of the plunger is connected to or provided on the plunger body, the visible marking may move together with the plunger body. As such, when the plunger body is for example axially moved within a barrel, the visible marking is also axially moved.

The marking of the plunger may for example be or comprise a line oriented generally perpendicular to the elongation direction of the plunger, which line may partially or preferably fully surround the plunger body. When the marking fully surrounds the plunger body, the marking may be visible from any viewing direction.

Additionally, or alternatively, the marking of the plunger may for example be or comprise one or more arrows, triangles, circles, other symbols, or any combination thereof.

A marking may be visible on the plunger by virtue of the marking having a different colour than sections of the plunger body axially adjacent to the marking. For example, the marking may have a generally dark colour, such as black. Sections of the plunger body axially adjacent to the marking may have a generally light colour, such as white, or may even be transparent.

Preferably, the plunger has a single marking, in particular a single marking visible through the barrel in use. This may prevent confusion for the user on which marking is to be used for visually determining a liquid volume present inside the barrel—i.e. which marking of the plunger has to be aligned with a marking of the barrel.

In general, the marking of the plunger may be a printed marking, for example printed onto the plunger body. Alternatively, the marking may be a connectable marking connectable to the plunger body. For example, the marking may be ring-shaped, or at least partially ring shaped. The marking may for example be clamped on the plunger, or glued onto the plunger.

As an even further option, the marking of the plunger may be formed by a different material than section of the plunger adjacent to the marking. The material may for example be different in that it has a different colour or general visual appearance such that a user can visually distinguish the marking of the plunger, in particular through a transparent part of a barrel.

Embodiments of the plunger may further comprise a thickened proximal end section, which may be used as a stop together with the barrel, which stop prevents the plunger from being inserted further into the barrel. The thickened proximal end section may thus determine a non-retracted position of the plunger. The thickened proximal end section may be thickened in the sense that it has a larger diameter than a distally adjacent section of the plunger. The thickened proximal end section may be provided at an opposite side of the plunger body as the piston.

A distance between the piston and the marking may be smaller than a distance between the thickened proximal end section and the marking, in particular at least two times smaller. Alternatively, a distance between the piston and the marking may be equal to or larger than a distance between the thickened proximal end section and the marking. The positioning of the marking of the plunger may for example depend on the presence of a label on the barrel body, which label may obstruct the view of a user into the barrel.

A second aspect provides a barrel for a syringe assembly. It will be understood that the plunger according to the first aspect may be used in conjunction with the barrel according to the second aspect, but conceivably also with other barrels.

The barrel according to the second aspect comprises a barrel body, with an axial passage therethrough, wherein the barrel body is at least partially transparent, and the barrel comprises a zero marking on the barrel body, which zero marking is positioned at a distance from a distal end of the barrel body.

When the zero marking of the barrel is positioned at a distance from a distal end of the barrel body, a part other than a piston of a plunger can be used to visually determine a volume of liquid present in a syringe assembly formed by the barrel according to the first aspect and a plunger, in particular to determine that this volume is essentially zero.

When the barrel body is at least partially transparent, objects inside the barrel body-such as a plunger, in particular a marking of the plunger—may be visible from outside the barrel body. Thus, in particular, the barrel body may be at least partially transparent for the marking of the plunger. Transparent may hence be interpreted as at least partially see-through. In general, the barrel body may consist of or comprise glass and/or a polymer such as polypropylene.

A zero marking may be defined as comprising any visual marking indicative to a user of essentially zero volume of liquid being present in the barrel. The zero marking may be used in conjunction with a marking on a plunger positioned inside the barrel. For example, at least part of the zero marking may resemble the number zero (“0”), the word zero, in any language, for example in combination with a quantity, such as 0 mL, or any other word indicative to a user of essentially zero volume of liquid being present in the barrel, such as the word empty, in any language, or any equivalent wording, in any language, or any symbol indicative to a user of essentially zero volume of liquid being present in the barrel, such as a letter indicative of the word empty, or an equivalent wording, for example the letter E for empty, or any combination thereof.

As an option, the barrel may comprise a connection flange with a skirt part, in particular for forming a Luer-lock connection with a needle hub. In such embodiments, the zero marking may be positioned at a distance from the connection flange, and the connection flange may be positioned between the distal end of the barrel body and the zero marking. The connection flange is in particular positioned at or near the distal end of the barrel body.

It will be understood that it may be impractical, or even impossible, to print a marking too close to the connection flange, as the radially extending connection flange may hinder a printing head. As such, with previously known barrels, no zero marking could or would be provided. With the barrel according the second aspect, a zero marking can now be provided on the barrel because the zero marking is positioned at a distance from the distal end of the barrel body.

A volume of the axial passage through the barrel may be smaller than 0.5 mL, in particular even smaller than 0.3 mL, or even smaller than 0.2 mL. As such, a maximum liquid capacity of the barrel may be smaller than 0.5 mL, in particular even smaller than 0.3 mL, or even smaller than 0.2 mL. It will be understood however that the present disclosure also encompasses barrels with other liquid capacities, also more than 0.5 mL, for example 1.0 mL.

Next to or as an alternative to the zero marking, as an option, one or more volume markings on the barrel body may be comprised by the barrel. The one or more volume markings are positioned at an axial distance from the distal end of the barrel body, and at an axial distance from the zero marking, if the barrel also comprises a zero marking. A volume marking may for example resemble a particular number or numbers, a number or numbers written as one or more words, in any language, for example in combination with a quantity, such as 0.05 mL.

Any marking on the barrel, such as a zero marking or volume marking, may comprise one or more alignment markings with which a marking of the plunger may be aligned. An alignment marking may for example be a line essentially perpendicular to the axial direction, which line may partially or fully surround the barrel body.

A third aspect provides a syringe assembly, comprising a barrel, for example according to the first aspect, and a plunger, for example according to the second aspect.

In the assembly, the plunger is arranged to be at least partially inserted into the axial passage of the barrel body, in particular with the piston first. The marking of the plunger is visible through the transparent part of the barrel body, and is as such visible for a user of the syringe assembly.

When an annular spacing is present between the marking of the plunger and an inner wall of the barrel body, the marking may not hinder the movement of plunger in the barrel body. Furthermore, if the marking would contact the inner wall—and thus no annular spacing would be present—the marking may increase undesired friction between the plunger and the barrel.

Alternatively, in assembled state of the syringe assembly, the marking of the plunger may contact an inner wall of the barrel body. For example, the marking may contact the inner wall of the barrel body over an entire circumference of the marking, or over only part thereof. In case the marking contacts the inner wall of the barrel body, the marking of the plunger may visually appear to have the same diameter as an outer diameter of the barrel due to refraction of light. This may make it more convenient for a user to align the marking of the plunger with a marking on the barrel.

The barrel of a syringe assembly may be filled with less than 1 mL, less than 0.5 mL, in particular less than 0.2 mL, or even less than 0.1 mL of liquid, in particular liquid medicament. In particular when the liquid medicament is for use in ophthalmology, the barrel may be filled with 0.1 mL or less, for example approximately 0.05 mL, of liquid medicament. A syringe assembly with any of these amounts of volume therein may be supplied as a prefilled syringe assembly—i.e. filled at a location different from the location where the liquid is expelled from the syringe assembly.

Embodiments of the syringe assembly may further comprise a needle hub with a needle, which needle hub is arranged to be connected to the barrel, at a distal end of the barrel. The connection may for example be a threaded connection, such as a Luer lock, or a fitted connection, such as a Luer slip, for example according to ISO 80369-7:2016 or 80369-7:2021.

The needle hub may comprise a pocket at a proximal end of the needle hub, for example upstream of the needle in use, which pocket is arranged for accommodating at least part of the piston of the plunger in the non-retracted state of plunger, and optionally also part of a distal end of the barrel, in particular of the barrel body.

For example when the plunger is arranged to be inserted into the barrel body at a proximal end of the barrel body, at least part of the piston of the plunger may extend out of the distal end of the barrel body, in particular at least in a non-retracted state of the plunger and optionally also in a partially—i.e. slightly-retracted state of the plunger. When the piston of the plunger can at least partially extend out of the distal end of the barrel body, all liquid present in the entire barrel body may be pushed out of the barrel body using the plunger.

When a negative of a shape of at least part of the pocket of the needle hub essentially corresponds to at least part of a shape of the piston of the plunger, the dead volume of the syringe assembly may be reduced or even eliminated. For example when the piston of the plunger has a convex or generally spherical shape, at least part of the pocket in the needle hub may have a concave or hollow shape. The pocket of the needle nub may be further shaped to accommodate part of a distal end section of the barrel body therein.

It will in general be understood that any embodiment of a syringe assembly disclosed herein may have a dead volume which is essentially 0 mL, essentially 0.0 mL, or even essentially 0.00 mL. This essential absence of a dead volume may for example be achieved by the shape of the piston, the shape of the barrel, and/or the shape of the needle hub. Any embodiment of the syringe assembly may as such be a syringe assembly for use in ophthalmology. In this field, accurate and/or precise dosing of liquid medicament is particularly important, as the volumes of liquid medicament administered are very small—e.g. less than 0.05 mL—to prevent excess pressure build-up in an eye of a patient. Furthermore, only 10% or less deviation of the desired dose may be allowed.

### BRIEF DESCRIPTION OF THE FIGURES

In the figures,

FIGS. 1A and 1B show an embodiment of a syringe assembly in an isometric view;

FIGS. 2A and 2B show the syringe assembly in a front view;

FIGS. 3A and 3B show the syringe assembly in a section view; and

FIG. 4 shows a detailed view of FIG. 3B.

### DETAILED DESCRIPTION OF THE FIGURES

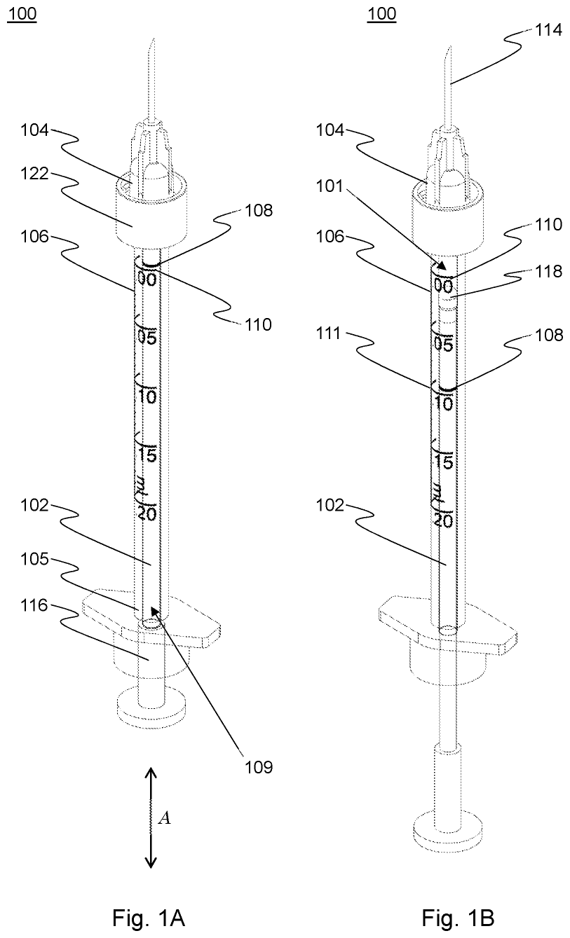

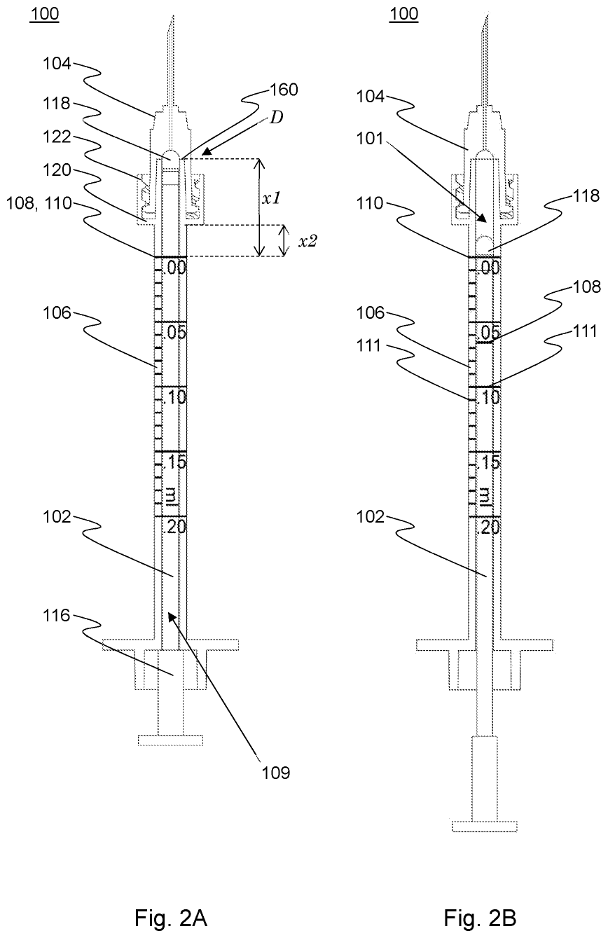

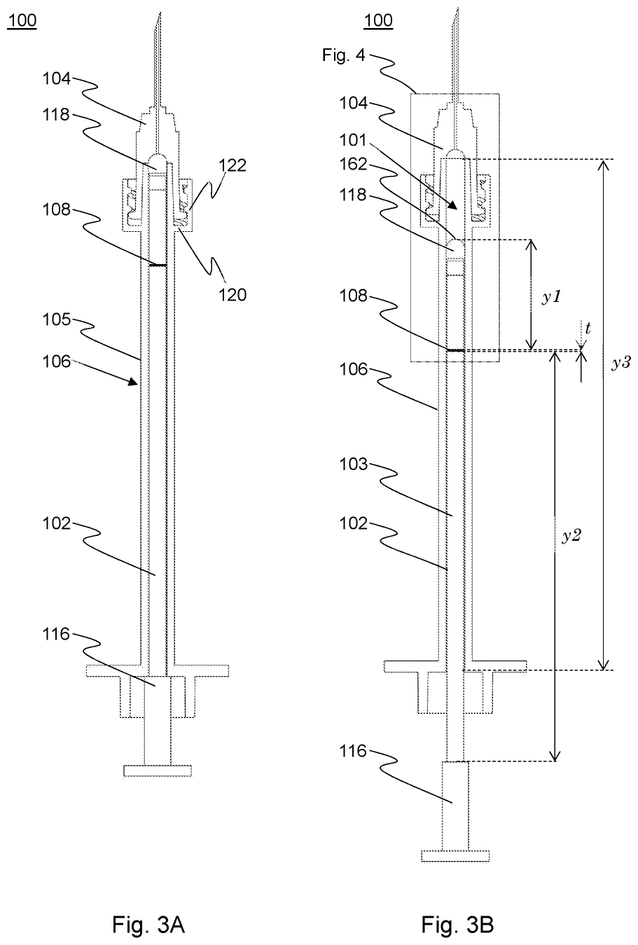

FIGS. 1A-3B show an embodiment of a syringe assembly 100, comprising a barrel 106, plunger 102, and needle hub 104. In FIGS. 1A and 1B, the syringe assembly 100 is shown in an isometric view, in FIGS. 2A and 2B, the syringe assembly 100 is shown in a front view, and in FIGS. 3A and 3B, the syringe assembly 100 is shown in a section view. Furthermore, as will be elaborated on below, in FIGS. 1A, 2A, and 3A, the plunger 102 is depicted in a non-retracted state, whereas in FIGS. 1B, 2B, and 3B, the plunger 104 is depicted in a partially retracted state.

In general, the same embodiment of a syringe assembly 100 is depicted in the figures. However, this should not be construed as if the scope of the present disclosure would be limited to this embodiment. The skilled person will appreciate that also further embodiments of the syringe assembly 100 are envisioned, in particular with any combination of options disclosed herein, in particular in conjunction with the barrel and the plunger. It will be understood that for clarity of the figures, not each component is provided with a reference number in each of the figures. An absent reference numeral does not imply absence of said component.

FIGS. 1A and 1B show the syringe assembly 100, comprising a plunger 102 which is inserted into a barrel 106, in particular in a hollow axial passage 109 of the barrel 106. In the partially retracted state of the plunger 102 inside the barrel 106, depicted in FIG. 1B, a liquid volume 101 is available which can be filled with a liquid, such a liquid medicament, in particular a liquid medicament for use in ophthalmology. The plunger 102 comprises a piston 118 at a distal end of the plunger, which piston 118 in use forms an essentially liquid-tight seal with an inner wall of the barrel.

As can be seen for example in FIGS. 1A and 1B, the marking 108 of the plunger 102 is visible from the outside through the barrel 106. In FIG. 1A, a user can thus visually determine that the marking 108 of the plunger 102 is aligned with an optional zero marking 110 on the barrel. In FIG. 1B, a user can visually determine that the marking 108 of the plunger 102 is aligned with a volume marking 111, which in this case is indicative of the liquid volume 101 available being approximately 0.10 mL.

In FIG. 1A, the plunger 102 is shown in a non-retracted state, in which a thickened proximal end section 116 of the plunger 102 may abut the barrel body 105. Additionally, or alternatively, the non-retracted state—i.e. the state wherein the plunger is inserted furthest into the barrel—may be determined by a shape of a needle hub, wherein the needle hub forms a stop for the plunger, in particular a stop for a piston of the plunger.

In use, the non-retracted state may correspond to a state of the syringe assembly 100 in which essentially no liquid is present in the barrel. By axially moving the plunger in the barrel body, the plunger may be moved into a retracted state, as for example shown in FIG. 1B. The axial direction is indicated in FIG. 1A with a double arrow marked A. By retracting the plunger 102 relative to the barrel 106, liquid may be sucked into the barrel 106, in particular into the liquid volume 101. By pushing the plunger 102 further into the barrel 106, the liquid in the liquid volume 101 may be discharged from the barrel 106.

As indicated in the figures, embodiments of the barrel 106 may comprise multiple markings 111, including an optional zero marking 110. The markings 111 may be numbers, such as 0.00, 0.05, 0.10, 0.15, 0.20, an indication of the quantity with which the numbers are used, such as mL, and one or more lines, which may or may not be read in conjunction with one of the numbers. For example, for each step of 0.05 mL, a longer line is provided, whereas for each step of 0.01 between the steps of 0.05 mL, a short line is provided. In other embodiments, other step sizes may be used, for example 0.002 mL or 0.001 mL.

When a barrel 106 comprises multiple markings, preferably, at least the part of the barrel body 105 between the multiple markings is at least partially transparent, such that a user can visually determine when a marking 108 of the plunger 102 is aligned with a marking of the barrel. As such, in the example of FIG. 2B, a user can visually determine that the liquid volume 101 amounts to approximately 0.065 mL. It will be understood that embodiments of the barrel 106 are also envisioned comprising a single marking, which may for example be indicative of a particular volume, such as 0.05 mL, or a single set of markings which together are indicative of a particular volume, such as 0.05 mL.

In FIG. 2A, a distance x1 is indicated, which is a distance between a distal end plane 160 of the barrel 106 and the most distal marking, which in this particular embodiment is a zero marking 110.

Also, a distance x2 is indicated in FIG. 2A, which is a distance between a connection flange 120 of the barrel 106 and the most distal marking. It may be preferred to minimise distance x2. However, the minimum value for distance x2 may for example be determined by the process with which the most distal marking is applied to the barrel 106, for example a printing process. It may be preferred not to print or provide any marking on outside of the skirt part 122, since it may be difficult to align a marking of the plunger with such a marking due to a radial distance between the skirt part 122 and the plunger.

FIGS. 3A and 3B show a section view of the syringe assembly 100, and as such it will be understood that markings on the barrel 106 are not visible in these figures. In FIG. 3B, a distance y1 is indicated, which is a distance between an outer distal end point 162 of the plunger 103 and the marking 108 of the plunger 103. As an option, this distance y1 is may be smaller than a distance y2 between the marking 108 and the thickened proximal end section 116 of the plunger 102. The distance y1 may even be less than 50% of distance y2, or even less than 25% of distance y2. Other embodiments are however envisioned in which the distance y1 is larger than the distance y2, for example due to the presence of a printed or glued label on the barrel body.

Additionally or alternatively, the distance y1 may be less than 50%, or even less than 25% of an axial length y3 of the axial passage of the barrel 106. As such, it may be prevented that the marking 108 is moved out of the barrel 106 when the plunger is in a retracted state. If the marking 108 of the plunger is outside the barrel 106, the marking 108 may not be useable any more for a user to visually determine the volume of liquid 101 inside the barrel.

A thickness t is also indicated in FIG. 3B, which resembles a thickness of the marking 108 of the plunger 102. The thickness t may be smaller than 20% of distance y1, smaller than 10% of distance y1, or even smaller than 5% of distance y1. The thickness t may generally correspond to a thickness of a marking of the barrel, for example a line as a marking of the barrel, or may have a smaller thickness than the marking of the barrel. As can be for example seen in FIG. 2A, this may allow the marking 108 of the plunger 102 to appear hidden behind the marking 110 of the barrel, which in turn may aid a user in properly aligning the markings.

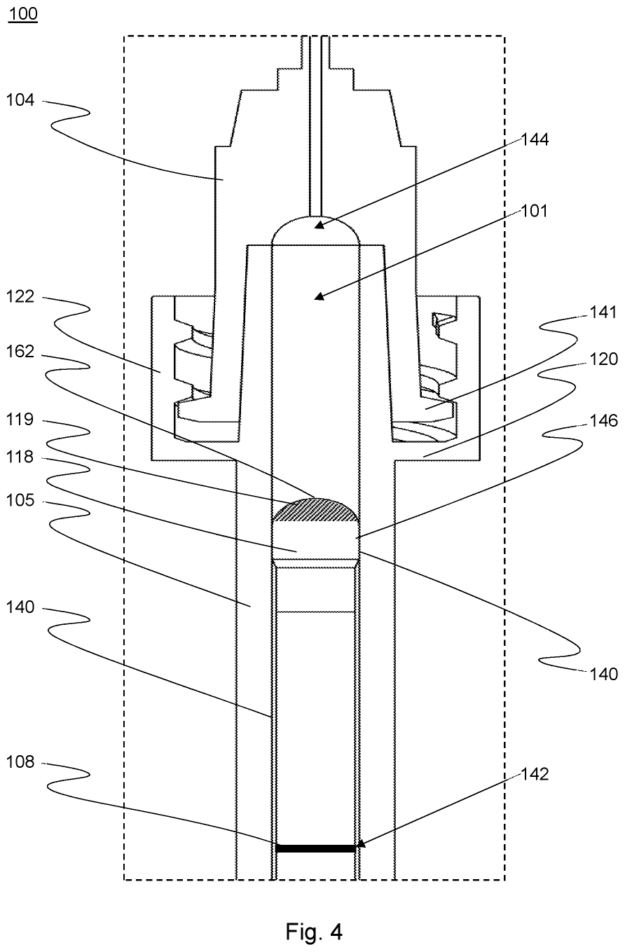

FIG. 4 shows a detailed view of the syringe assembly 100 in FIG. 3B. As can be seen in FIG. 4, the needle hub 104 may comprise a connection flange 141, which can be threaded into the skirt part 122 of the barrel 106, for example according to the Luer-lock standard. In general, the skirt part 122 may be generally axially oriented, and the connection flange 120 may be generally radially oriented.

In FIG. 4, an example of a distal end section 119 of the piston 118 of the plunger 102 is shown hatched. FIG. 4 shows an example of a curved distal end section 119 of the piston 118. In general, a distal end section of the piston may have a flat end-plane, or may have a curved end-plane. In particular, the curved end-plane may be a convex end-plane, for example a generally dome-shape end-plane as shown in FIG. 4. Such a convex end-plane may prevent liquid from becoming trapped in the syringe 100, in particular in combination with the needle hub 104 as will be explained below. Additionally or alternatively, the convex end-plane may prevent air from becoming trapped between the end-plane and the needle hub, for example when the plunger is inserted into the barrel and fully pressed down into the non-retracted state.

In general, for syringe assemblies, it may be preferred to reduce a dead volume of the syringe, or even to eliminate the dead volume. The dead volume is defined as the volume of liquid trapped in the syringe assembly even when the plunger is fully inserted into the barrel—in the non-retracted state as for example depicted in FIG. 3A. In particular when the syringe assembly is used in conjunction with expensive liquids, such as liquid medicaments, in particular liquid medicaments for ophthalmology, even a small reduction in dead volume may be economically beneficial as it reduces a volume of liquid medicament which has to be discarded together with the syringe assembly after the rest of the liquid medicament has been discharged from the syringe assembly. Additionally or alternatively, a reduction or elimination of dead volume may increase the precision and/or accuracy with which a volume of liquid can be discharged from the syringe assembly.

In the embodiment depicted in FIG. 4, in order to reduce or even eliminate the dead volume, the needle hub 104 comprises a pocket 144 at a proximal end of the needle hub. The pocket 144 is arranged for accommodating at least part of the piston 118, in particular the distal end section 119 of the piston 118 in the non-retracted state of plunger 102. A negative of a shape of at least part of the pocket 144 preferably essentially corresponds to a shape of the distal end section 119 of the piston, as can be seen for example in FIG. 4. In order for the piston 118 to be inserted at least partially into the pocket 114 of the needle hub 104, at least part of the piston 118 may extend out of the distal end of the barrel 106 when the plunger is in the non-retracted state. This state is for example visible in FIG. 3A. The non-retracted state may correspond to a fully inserted state of the plunger. When positioning the marking 108 on the plunger, due account may have to be taking for how far the piston 118 may extend out of the distal end of the barrel 106 in the non-retracted state.

A sealing section 146 of the piston 118 is arranged to in use form a liquid-tight seal with an inner wall 140 of the hollow passage through the barrel body 105. The sealing section 146 may be positioned adjacent to the distal end section 119 of the piston 118, and in between the distal end section 119 and the marking 108.

As an option depicted in FIG. 4, an annular spacing 142 may be present between the marking 108 of the plunger and the inner wall 140 of the barrel body 105. As such, in use, when moving the plunger in the barrel, the marking 108 may not hinder this movement. In alternative embodiments, the marking 108 may contact the inner wall 140 of the barrel body 105.

In the description above, it will be understood that when an element is referred to as being connect to another element, the element is either directly connected to the other element, or intervening elements may also be present. Also, it will be understood that the values given in the description above, are given by way of example and that other values may be possible and/or may be strived for.

It is to be noted that the figures are only schematic representations of embodiments that are given by way of non-limiting examples. For the purpose of clarity and a concise description, features are described herein as part of the same or separate embodiments, however, it will be appreciated that the scope of the disclosure may include embodiments having combinations of all or some of the features described.

The word ‘comprising’ does not exclude the presence of other features or steps. Furthermore, the words ‘a’ and ‘an’ shall not be construed as limited to ‘only one’, but instead are used to mean ‘at least one’, and do not exclude a plurality.

1. A plunger for a syringe assembly, the plunger comprising:
an axially elongated plunger body with a piston at or near a distal end of the plunger;
a visible marking connected to or provided on the plunger body;
wherein the visible marking is positioned at a distance of the piston.

2. The plunger according to claim 1, wherein the piston has a curved end section.

3. The plunger according to claim 1, wherein the plunger comprises a single marking.

4. The plunger according to claim 1, wherein the marking is a printed marking.

5. The plunger according to a claim 1, wherein the marking is a connectable marking connectable to the plunger body.

6. The plunger according to claim 1, further comprising a thickened proximal end section, and wherein the distance between the piston and the marking is smaller than a distance between the thickened proximal end section and the marking.

7. A syringe assembly, comprising:
a barrel, comprising a barrel body with an axial passage therethrough, wherein the barrel body is at least partially transparent and the barrel comprises at least one marking;
a plunger according to claim 1, which plunger is arranged to be at least partially inserted into the axial passage of the barrel body;
wherein the marking of the plunger is visible through at least part of the barrel body.

8. The syringe assembly according to claim 7, wherein in assembled state, an annular spacing is present between the marking of the plunger and an inner wall of the barrel body.

9. The syringe assembly according to claim 7, wherein in assembled state, the marking of the plunger contacts an inner wall of the barrel body.

10. The syringe assembly according to claim 7, further comprising a needle hub with a needle, which needle hub is arranged to be connected to the barrel, at a distal end of the barrel.

11. The syringe assembly according to claim 7, wherein the barrel is filled with less than 1 ml of liquid medicament for use in ophthalmology.

12. The syringe assembly according to any claim 7, wherein the plunger is arranged to be inserted into the barrel body at a proximal end of the barrel body, and at least part of the piston of the plunger can extend out of the distal end of the barrel body in a non-retracted state of plunger.

13. The syringe assembly according to claim 12, further comprising a needle hub with a needle, which needle hub is arranged to be connected to the barrel, at a distal end of the barrel, wherein the needle hub comprises a pocket at a proximal end of the needle hub, which pocket is arranged for accommodating at least part of the piston of the plunger in the non-retracted state of the plunger.

14. (canceled)

15. The syringe assembly according to claim 7, wherein the marking of the plunger is aligned with the at least one marking of the barrel, when the plunger is fully inserted into the barrel and a dead volume of the syringe assembly is 0 mL.

16. The syringe assembly according to claim 7, wherein the marking of the plunger is aligned with at least one marking of the barrel when 0.05 mL or less of volume is available for liquid in the barrel, distally from the piston of the plunger.

17. The syringe assembly according to a claim 7, wherein the barrel comprises a connection flange with a skirt part at a distal end of the barrel, and wherein the marking of the plunger is misaligned with the skirt part when the plunger is fully inserted into the barrel.

18. (canceled)

19. A barrel for a syringe assembly, the barrel comprising a barrel body, with an axial passage therethrough, wherein:
the barrel body is at least partially transparent; and
the barrel comprises a zero marking on the barrel body, which zero marking is positioned at a distance from a distal end of the barrel body.

20. The barrel according to claim 19, further comprising:
a connection flange with a skirt part,
wherein the zero marking is positioned at a distance from the connection flange, and the connection flange is positioned between the distal end of the barrel body and the zero marking.

21. The barrel according to claim 19, wherein a volume of the axial passage through the barrel is smaller than 0.5 mL.

22. (canceled)
